(19) United States
(12) Patent Application Publication
(10) Pub. No.: US 2003/0143759 A1
(43) Pub. Date: Jul. 31, 2003

(54) Assays for determining anticoagulant cofactor activity

(76) Inventor: Bjorn Dahlback, Malmo (SE)

Correspondence Address: TESTA, HURWITZ & THIBEAULT, LLP, HIGH STREET TOWER, 125 HIGH STREET, BOSTON, MA, 02110, US

(21) Appl. No.: 09/912,947

(22) Filed: Jul. 25, 2001

Related U.S. Application Data
(62) Division of application No. 08/500,917, filed on Oct. 20, 1995.

(51) Int. Cl. G01N 33/537; A61K 38/36

(52) U.S. Cl. 436/538; 514/21

(57) ABSTRACT

Methods are disclosed for determining, in a sample derived from a human, the functional activity of a component of the human blood coagulation system, which activity can be correlated to conversion of a substrate specific for activated Protein C (APC), by measuring in an assay medium containing the sample and a substrate for APC, the conversion of the substrate by APC and correlating the conversion to the functional activity of the component. When the component is anticoagulant Factor V, at least one of exogenous APC, Protein S or an inhibitor of Protein S activity is added to the medium. When the component is Protein C, APC, or Protein S, exogenous anticoagulant Factor V or an inhibitor of anticoagulant activity of Factor V is added to the medium. Methods are also disclosed for diagnosing a blood coagulation/anticoagulation disorder or for determining a predisposition thereto in a human by determining anticoagulant Factor V activity in an assay medium containing a sample derived from the human.

The present invention is generally related to a novel anticoagulant cofactor activity involved in the human blood coagulation system and possibly also involved in the blood coagulation system of some other mammal species.

Blood coagulation is a complex system involving a large number of proteins that function in concert with platelets to yield hemostasis. The coagulation system is strictly regulated by a series of anticoagulant proteins present in plasma and on the surface of endothelial blood cells (Esmon, J. Biol. Chem. 264 (1989) 4743-4746; Bauer, Sem. Hematol. 28 (1991) 10-18; and Rapaport, Blood 73 (1989) 359-65). Under physiological conditions, pro- and anti-coagulant mechanisms are delicately balanced to provide hemostasis and coagulation. Disturbances in this balance result in either bleeding or thromboembolic disorders.

The present invention is related to a novel activity involved in a physiologically important anticoagulant system associated with Protein C and Protein S that has been elucidated in recent years and is shown below as part of the blood coagulation interactions illustrated in the following Scheme 1.

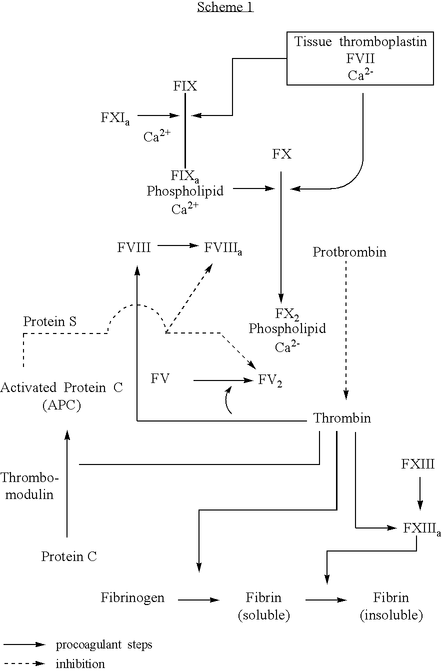

In the above mentioned anticoagulant system, Protein C, a vitamin K-dependent plasma protein, is a key component that after activation to Activated Protein C (APC) on endothelial cells by the thrombin/thrombomodulin complex selectively degrades the coagulation Factors Vₐ and VIIIₐ, i e the activated forms of the coagulation Factor V and VIII, respectively. (Esmon, loc. cit.; Stenflo, in Protein C and related proteins, ed. Bertina (Churchill Livingstone Longham Group, UK) (1988) 21-54; Mann et al., Ann. Rev. Biochem. 57 (1988) 915-956; and Kane et al., Blood 71 (1988) 539-55).

The activity of APC is influenced by another vitamin K-dependent plasma protein, designated Protein S, which functions as a cofactor to APC in the degradation of Factors Vₐ and VIIIₐ, (Esmon, loc. cit.: Stentlo, loc. cit.; and Dahlbäck, Thromb. Haemostas. 66 (1991) 49-61).

The above mentioned Factors Vₐ and VIIIₐ are phospholipid-bound cofactors involved in the activation of Factor X and prothrombin, respectively, and are, thus, indirectly involved in the conversion of fibrinogen to fibrin, i e in clot formation. Accordingly the rate of the coagulation reaction is dependent on the balance between the activation of Factors VIII and V and the degradation of their activated forms, the unactivated Factors VIII and V being poor substrates for APC.

Disturbances in the blood coagulation system are frequently manifested as serious and often life-threatening conditions and knowledge about the underlying causes for the disturbances is often crucial in order to enable diagnosis and/or successful therapy of a manifested disease or the screening of individuals having a predisposition for a blood coagulation disease. For instance, therapeutic use of purified Protein C has been developed as a result of the discovery of Protein C deficiency associated with thrombophilia.

Thrombophilia can be defined as a tendency towards early-onset venous is thomboembolic disease in adults in the absence of known risk factors. Although abnormalities have been determined for some thrombophilic patients, in the majority of such cases no laboratory test abnormalities were identified.

The present invention is related to a new defect in anticoagulant response to activated Protein C, called APC-resistance, which has been shown to be inherited and associated with familial thrombophilia.

In a few cases thrombophilia has been associated with hypothetical factors, such as an anti-Protein C antibody (Mitchell et al, New England Journal of Medicine, 1987, Vol. 316, 1638-1642), an anti-cardiolipin antibody (Amer et al, Thrombosis Research 57 (1990) 247-258) and a defect Factor VIII molecule (Dahlbäck et al, Thromb Haemost 65, Abstract 39, 658 (1991)).

In WO 93/10261, which is a reference published after the earliest priority date claimed for the present application, in vitro methods for the diagnosis of a manifested blood coagulation disorder or for the screening of individuals being predisposed for a blood coagulation disorder are disclosed. These methods are based on measurement of the anticoagulant response to exogenous APC added to a plasma sample from the individual to be tested, a weak anticoagulant response to APC, i e APC-resistance, indicating manifestation of or predisposition for blood coagulation disorders, and especially a thromboembolic disease. No explanation for APC-resistance is given but the resistance to APC is suggested to be due to unknown interactions in the blood coagulation system or to unknown coagulation factor(s) thereof. However, several possible explanations connecting the APC-resistance to functional Protein S deficiency, a Protein C inhibitory antibody, a protease inhibitor for APC or a mutation giving a APC-resistant Factor Vₐ molecule or a Factor VIII gene mutation were ruled out.

According to the present invention it has been found that APC-resistance is due to deficiency of a previously unrecognized anticoagulant cofactor activity enhancing the proteolytic effect of APC directed against Factor Vₐ and Factor VIIIₐ. The findings that form the basis for the discovery of the present anticoagulant coagulant cofactor activity have been reported in Dahlbäck et al. Proc. Natl. Acad. Sci. USA, 90 (1993) 1004-1008, said reference having a publication date after the earliest priority date claimed for the present application.

More specifically, this anticoagulant activity has been found to be expressed by Factor V, a finding that is quite surprising, since Factor V is the precursor to the procoagulant Factor Vₐ, the latter being degraded by APC in the above mentioned Protein C anticoagulant system. Thus, factor V is the second cofactor that has been found for APC, the first one being Protein S is mentioned above. Accordingly, the present novel anticoagulant cofactor activity is designated “APC-cofactor 2 activity” or “Factor V anticoagulant activity” and, where appropriate, Factor V is also designated “APC-cofactor 2”. The prior known activity of Factor V is designated “Factor V procoagulant activity”. However, the possibility that the said activity is associated with Factor Vₐ cannot be ruled out entirely

The discovery of the novel anticoagulant cofactor activity according to the present invention is based on the discovery of one patient with thrombosis and an abnormal APC-resistance when his plasma was assayed with the methods disclosed in the above-mentioned WO 93/10261 (with a priority date of Nov. 13, 1991, US being one of the designated states; the disclosure of said reference is incorporated herein by reference) and by Dahlbäck et al (Thromb Haemost 65, Abstract 39 (1991) 658). When studying a large cohort of patients thrombosis APC-resistance was found to be the underlying cause in 30-40% of idiopathic thromboembolic events (Thromb Haemostas 69, 999, abstract (1993)).

Later, it has been found according to the present invention that a crude fraction obtained from normal plasma contained an activity, which corrected the defect of APC-resistant plasma, whereas the corresponding fraction from APC-resistance plasma from a patient with pronounced APC-resistance was inactive. This proves the existence of a novel cofactor to APC. In addition, by using preparations purified in the said activity in assays, which have been designed to measure this activity, conclusive evidence for the existence of a novel cofactor to APC has been achieved.

According to the present invention it has, thus, surprisingly been found that human Factor V has activity as a cofactor to APC in addition to its well known function as a precursor to the procoagulant Factor Vₐ. Possibly this dual function of human Factor V is also expressed by Factor V derived from blood from some animals species, especially mammals, but not expressed in other species. For instance, all results so far obtained indicate that bovine plasma is lacking the said activity.

The said cofactor activity of Factor V means that it enhances the proteolytic effect of activated Protein C, thus promoting the degradation of Factor Vₐ, i e the activated form of Factor V, as well as the degradation of Factor VIIIₐ.

It is previously well known that the procoagulant activity of Factor V is due to its activation by thrombin, three peptide bonds being cleaved resulting in the formation of the procoagulant Factor Vₐ as a complex between the N- and C-terminal portions of the native Factor V. The function of the two large activation peptides derived from the central portion of Factor V is, however, unknown. As will be shown in the experimental part of this disclosure, the APC-cofactor 2 activity has not been found for Factor Vₐ in the APC-resistance test used.

Thus, the APC-cofactor 2 activity is preferentially expressed by the intact Factor V molecule, probably the large fragments cleaved off during activation thereof to Factor Vₐ contributing to a major part of said activity. However, the possibility that the said activity is associated with a molecular entity which forms a highly stable complex with Factor V which is not split under the purification procedures used to isolate the Factor V having APC-cofactor 2 activity cannot be ruled out entirely. Accordingly, in connection with the present invention the expressions “Factor V” and “Factor V having APC-cofactor 2 activity” and the like are intended also to encompass said complex of Factor V and also fragments of Factor V, preferably other than the fragments originating from thrombin cleavage of Factor V, having the said activity. Modified Factor V with retained APC-cofactor activity may also be obtained through proteolytic cleavage by other enzymes of human or non-human origin such as snake venom enzymes and other proteases. Furthermore, the APC-cofactor 2 activity was found to remain after partial proteolysis by unknown enzyme during purification thereof, indicating a potential existence of APC-cofactor 2 active Factor V fragments. The expressions APC-cofactor 2 as well as Factor V having anticoagulant activity include fragments and subunits of Factor V/Vₐ expressing the activity or an immunologic determinant related to a region associated with the said activity. Although for the sake of convenience coagulation factors and the like are not species related throughout this description such factors of human origin are preferably intended unless otherwise specified.

In the experimental part of this disclosure the procedures used for purification and characterization of the present novel APC-cofactor 2 activity is described and its connection with Factor V is verified.

In summary, the evidences for the presence of the APC-cofactor 2 activity on Factor V are:

1. The procedure designed for the isolation of APC-cofactor 2 activity and earlier methods for isolation of Factor V are very similar. On SDS-PAGE three bands appear at approximately 200-220 kDa (C-terminal portion), 140-160 kDa (N-terminal portion) and 330 kDa which also is very similar to what has been reported for Factor V. (Cf the experimental section of the disclosure and Dahlbäck et al, J. Clin. Invest. 66 (1980) 583-91.) The intensity of the band at 330 kDa is enhanced for both APC-cofactor 2 activity and Factor V when higher concentrations of protease inhibitors are used during the purification procedure. For instance, a benzamidine hydrochloride concentration of 10 mM gives rise to a significant band at 330 kDa.

2. Specific polyclonal antiserum against human Factor V (Dakopatt A/S, Denmark) reacts with each of the three bands associated with APC-cofactor 2 activity in Western blotting.

3. After addition of thrombin to the present preparations comprising APC-cofactor 2 activity the three bands disappear and the products obtained become indistinguishable from the products formed by thrombin activation of Factor V.

4. Seventeen monoclonal antibodies reacting with Factor V have been obtained by using a preparation purified in respect of APC-cofactor 2 activity as immunogen. Two of the monoclonal antibodies partially inhibited APC-cofactor 2 activity without inhibiting Factor V procoagulant activity.

5. Factor V procoagulant activity and APC-cofactor 2 activity are coeluted on every chromatographic material tested, Heparin Sepharose, Blue-Sepharose, Wheat Germ Lectin Sepharose, Q-Sepharose and S-Sepharose (Pharmacia, Sweden) illustrating materials that have been tested.

6. Both Factor V procoagulant activity and APC-cofactor 2 activity are retained on a matrix carrying polyclonal antibodies against human Factor V (Dakopatts A/S, Denmark).

7. Both Factor V procoagulant activity and APC-cofactor 2 activity are retained on matrices, such as Sepharose and Affigel, carrying antisera against different fragments of bovine Factor V, which cross-react with human Factor V.

8. Both Factor V procoagulant activity and APC-cofactor 2 activity are retained and coeluted on a chromatographic support, such as Affigel, carrying a high affinity monoclonal antibody, which had been prepared by using a preparation purified in respect of APC-cofactor 2 activity as immunogen. In itself, this antibody inhibited neither APC-cofactor 2 activity nor Factor V procoagulant activity. Elution was performed at a pH of approximately 10.5-11.

9. A recent publication disclosing that autoantibodies against Factor V may result in thrombosis (Kapur A et al, A.J. Hematol. 42 (1993) 384-388).

Preparations enriched in APC-cofactor 2 activity have been obtained by the same methods as have been used previously for the isolation of Factor V. It has been found that divalent metal ions, such as calcium ion, have a stabilizing effect on the APC-cofactor 2 activity and, hence, calcium ions X ere added during the purification.

Essentially the same purification procedure has been used as a first attempt in order to elucidate the novel activity disclosed in the above mentioned WO 93/10261. According to the results presented herein, the novel activity has been identified as a cofactor activity to APC expressed as a novel property of Factor V, or, possibly, a complex or fragments thereof as discussed above. Thus, alternative and simpler preparation methods will become available. Current methods, such as gel chromatography, affinity chromatography with e.g. anti-APC-cofactor 2 activity antibody as affinity ligand, ion exchange chromatography, etc, have been used, suitably after improvement. In addition, methods based on DNA-recombinant technique may be applicable.

Accordingly, the present invention is also related to a preparation derived from blood or blood related products, such as plasma, said preparation being purified in respect of a blood coagulation component which can express anticoagulant activity as a cofactor to APC thereby enhancing its proteolytic activity, directed against Factor Vₐ and Factor VIIIₐ, said blood coagulation component being comprised of Factor V or, optionally, a stable complex of Factor V and a molecular entity, which can express said activity.

The normal plasma level of Factor V is approximately 10-20 μg/ml. In analogy with other blood coagulation/anticoagulation factors, the APC-cofactor 2 activity in 1 ml normal plasma is arbitrarily designated 1 unit (U).

The present invention is also concerned with antibodies and antibody preparations specific for a region of Factor V that is associated with APC-cofactor 2 activity , i.e. a region in which there is a site carrying an epitope either causing APC-cofactor 2 activity or APC-cofactor 2 inactivity. Such antibody preparations may be polyclonal, or preferably, monoclonal. Preferably, the antibodies of such preparations bind specifically to one or more Factor V sites associated with APC-cofactor 2 activity. Alternatively, such a site could comprise an epitope involved in APC-cofactor 2 inactivity of Factor V and, thus, in APC-resistance. In connection with this invention the expression “epitope involved in APC-cofactor 2 inactivity” is meant to include an epitope related to decrease or loss of APC-cofactor 2 activity.

Polyclonal antibodies can be obtained in accordance with known methods comprising immunization of suitable animals, such as mouse, rat, rabbit, dog, horse, sheep, goat, birds, e.g. hen, chicken, etc, with a proper immunogen and recovery of the present antibodies from an appropriate fluid derived from said animal, e.g. from blood or serum in the case of mammals, or from eggs, when birds are immunized.

Preferably, the present antibodies are monoclonal antibodies which may be obtained by conventional methods, e.g. essentially as disclosed by Köhler, G. and Milstein, C., Nature 256, 495 (1975). Generally, a method to prepare monoclonal antibodies of the present invention includes immunizing a mammal, preferably a mouse, with a proper immunogen, producing hybridic cells by fusion of lymphocytes, such as splenic cells, from the immunized mammal with myeloma cells, selecting fused cells in a suitable medium, screening antibody-producing cells, cloning antibody-producing cells, i e hybridoma, and producing monoclonal antibodies in ascitic fluid of mice or, optionally, in a culture medium by propagation of the hybridoma therein. However, the present monoclonal antibodies, and fragments thereof binding to antigen, can also be obtained according to the methods based on recombinant technology, as is well known in this art. In such methods suitable host cells of eucaryotic or procaryotic origin can be used. Such host cells are well known in this field of the art.

As immunogen, a purified preparation of Factor V can be used or fragments and derivatives thereof comprising the antigenic determinants responsible for expression of APC-cofactor 2 activity. Such fragments or derivatives may be conjugated to an immunogenic carrier, usually a protein, to become antigenic.

By using as the immunogen, human Factor V deficient in APC-cofactor 2 activity (which can be obtained as described below) combined with a two step screening procedure for selecting hybridomas producing monoclonal antibodies reactive with the immunogen but not with normal intact human Factor V, monoclonal antibodies reacting specifically with a human APC-cofactor 2 inactivity epitope, i.e. an epitope related to decrease or loss of APC-cofactor 2 activity, may potentially be obtained.

A preferred embodiment of the present invention is related to monoclonal antibodies that bind to and also inhibit APC-cofactor 2 activity of Factor V, at least in part. The present invention is also related to derivatives and fragments of such monoclonal antibodies, which are able to bind to antigens.

According to the present invention, monoclonal antibodies produced by mouse/mouse hybridoma are preferred, since these are simple to obtain. Illustrative for such monoclonal antibodies are those produced by a novel hybridic cell line deposited on Dec. 8, 1993 in the PHLS Centre for Applied Microbiology & Research, European Collection of Animal cell culture, Salisbury, Great Britain with the provisional accession number XAM-4-5-1 93120846. In connection with the present invention monoclonal antibodies produced by this hybridoma are designated M4 (Master 4).

If not otherwise specified, the term “antibody (or antibody preparation)” encompasses the intact antibody with its two heavy and two light chains as well as different forms of derivatized antibodies containing the variable domains (Fᵥ), e g fragments such as Fab, Fab′, F(ab′)₂; single chain antibodies; labelled antibodies, such as radiolabelled, fluorescent or enzyme-coupled antibodies; antibodies bound to solid phases, etc.

A further embodiment of the present invention is concerned with antibody preparations, which comprise a definite number of monoclonal antibodies of the above-mentioned specificity, such as 1, 2, 3, 4, 5 or more different monoclonal antibodies, or are polyclonal. Polyclonal and monoclonal antibody preparations directed specifically against epitopes uniquely present in a site associated with APC-cofactor 2 activity are potentially useful in immunoassays for specifically determining the presence or absence of APC-cofactor 2 activity in a sample (quantitatively and qualitatively).

The present invention is also related to hybridomas that produce the monoclonal antibodies of the present invention, and preferably to the above mentioned hybridoma having the provisional accession number XAM -4-5-1 93120846.

Although polyclonal, and also monoclonal, antibodies specific for Factor V, that can be used to purify Factor V, are previously known, monoclonal antibodies deliberately raised against a region of Factor V associated with APC-cofactor 2 activity have not been disclosed before.

The antibody preparation (monoclonal as well as polyclonal) of the present invention may in most cases be used in purification procedures based on affinity chromatography in which antibodies of this invention are attached to a solid carrier and used to selectively bind Factor V present e.g. in a plasma preparation. Subsequently, Factor V, that has bound to the solid carrier, is eluted and collected.

The preferred monoclonal antibodies of this invention that bind to Factor V and inhibit, at least in part, APC-cofactor 2 activity of Factor V, can be used to inhibit said activity of Factor V. Such monoclonal antibodies may like the previously known anti Factor V antibodies, also be used to obtain plasma preparations deficient in APC-cofactor 2 activity.

Important aspects of the present invention are concerned with therapeutic methods, medicaments and pharmaceutical preparations, for which the knowledge of the novel anticoagulant activity of Factor V, i.e. APC-cofactor 2 activity, is used.

Accordingly, the present invention is also related to the use of Factor V, subunits or fragments thereof having anticoagulant activity as cofactor to APC for the manufacture of a medicament or pharmaceutical preparation intended for enhancing or restoring anticoagulant activity of APC in vivo. Specifically, such preparations are intended for treatment of patients suffering from, or predisposed for, vascular diseases, such as thromboembolic disorders including thrombosis and disseminated intravascular coagulation (DIC).

Such a medicament or pharmaceutical preparation may be comprised of a highly purified preparation of Factor V, which can be stored at low temperatures, such as −70° C.

The present preparations may also be used in connection with other conditions or situations in which an individual would benefit from a corrected or enhanced blood anticoagulant activity, for instance, in various clinical situations that are associated with increased risks for arterial and venous thrombosis.

Moreover, since the present APC-cofactor 2 activity is crucial for the effect of APC, this activity may be used per se or in combination with Protein C/APC and/or Protein S. Clinical situations where this may prove to be important include patients being deficient in APC-cofactor 2 activity, in particular in situations increasing the risks for thrombosis. In addition, supplemental APC-cofactor 2 activity may be beneficial in connection with myocardial infarction after thrombolytic therapy, in the post-operative period—in particular in risk patients as an adjuvant to patients treated for thrombosis, in patients undergoing microsurgery, etc.

The administration route for APC-cofactor 2 activity is that normally applied for therapy with blood coagulation/anticoagulation factors, such as intravenously or intraarterially injection or infusion. As has been suggested for other blood factors, oral administration can not be excluded. The amount to be administered shall be effective in the sense that it at least for a period of time fully or partially restores the effect of the patient's own activated Protein C or of coadminstered Protein C/activated Protein C, with the understanding that even smaller effects may be beneficial to a patient at risk for thrombosis. An amount in the range of 1-100, possibly 40-70, mg/day, can be assumed to be useful. Repeated administration is preferred, because Factor V expressing APC-cofactor 2 activity is metabolized in the mammalian body.

The different types of pharmaceutical compositions available are the same as in use for other blood coagulation/anticoagulation factors, but adapted to the specific stability requirements that are necessary for Factor V having APC-cofactor 2 activity. For instance lyophilized or spray dried powders, optionally diluted with appropriate vehicles, as well as sterile or aseptically produced aqueous solutions can be used.

A further aspect of the present invention is related to the use of Protein C/activated Protein C and/or Protein S for the manufacture of a pharmaceutical composition for the treatment of disorders related to deficiency in APC-cofactor 2 activity. The same types of compositions as intended for the prior art therapeutic use of Protein C and Protein S are applicable.

Another aspect of the present invention is related to a Factor V preparation deficient in APC-cofactor 2 activity and is preferably derived from humans. A potential therapeutic use thereof is in cases where an increase in Factor Vₐ activity over APC-cofactor 2 activity is beneficial to a patient.

The above-mentioned therapeutic methods and preparations are intended for mammals, particularly humans.

The novel anticoagulant cofactor activity according to the present invention can be used to develop methods for diagnosing such blood coagulation/anticoagulation disorders that are related to the functional activity of APC and also to develop methods for monitoring and/or measuring functional activities of components involved in the blood coagulation/anticoagulation system, that are directly or indirectly depending on the functional activity of APC.

Accordingly, a suitable embodiment of the present invention is related to a method for diagnosing a blood coagulation/anticoagulation disorder, preferably a thromboembolic disorder, or determining predisposition therefor, in an individual, preferably a mammal, such as a human being, said method comprising determining in a sample, preferably a blood or blood derived sample, such as plasma, derived from said individual, the level of a blood component expressing anticoagulant activity, said blood component being comprised of Factor V, the level of its anticoagulant activity as a cofactor to APC, being determined, an abnormal, preferably a decreased, level indicating manifestation of or predisposition for said disorders, in particular for a decreased level said disorder being a thromboembolic disorder.

Suitable embodiments of the above method are related to assaying the appropriate sample from an individual for Protein C/APC. Protein S or APC-cofactor 2 activity, and relating a found abnormal level, preferably a lowered level, to a diagnosis that the individual has a blood coagulation disorder related to the assayed factor, i.e. to activated protein C/Protein C, Protein S, or Factor V in its capacity as APC-cofactor 2, which defect may be an underlying cause for a thromboembolic disorder, or predispose for said disorder.

In the above methods the level of the anticoagulant APC-cofactor 2 activity is preferably measured in accordance with methods developed according to the present invention for assaying functional APC-cofactor 2 activity that are described below. Immuno-based activity assays and non-functional assays specific for Factor V carrying structural elements associated with its APC-cofactor 2 activity can also be used.

Thus, further aspects of the present invention are related to functional assays for activated Protein C/Protein C, Protein S and Factor V expressing APC-cofactor 2 activity and also to immune assays and nucleic hybridization assays for Factor V expressing APC-cofactor 2 activity, DNA and RNA sequencing methods.

The assays as such may have other uses than as diagnostics, for instance monitoring purification procedures of components in the APC-cofactor system, standardising control plasmas, etc.

A. Functional assays of APC, Protein C, APC-cofactor 2 activity and Protein S

These assays utilize similar protocols as described earlier (Bertina et al., Res. Clin. Lab. 20 (1990) 127-138; Wolf et al., Thromb. Haemost. 62 (1989) 1144-1145; WO-A-9102812; WO-A-9101382; WO 93/10261, the US designation of which is hereby incorporated by reference; Dahlbäck et al., Thromb. Haemost. 65, Abstract 39, (1991) 658). Thus, a component in the system of APC, Protein S and Factor V, the latter in its capacity as APC-cofactor 2, is assayed from the conversion of the appropriate APC substrate by APC. Normal APC substrates are Factors Vₐ and/or VIIIₐ, one or both of which preferably are added to the assay medium as enriched, or highly purified preparations, including preparations by recombinant technology, of unactivated (Factor V, Factor VIII) or activated proteins. Within a series of samples that are to be compared, the assay media have essentially the same levels of:

(a) at least one of Factor V having APC-cofactor 2 activity or an inhibitor that blocks the same sample derived activity and Protein S, or an inhibitor that blocks sample derived Protein S activity when APC or Protein C is to be assayed;

(b) at least one of Protein S or an inhibitor that blocks sample derived Protein S activity and APC, when APC-cofactor 2 activity is to be assayed; and

(c) at least one of Factor V providing APC-cofactor 2 activity or an inhibitor that blocks the same sample derived activity and APC, when Protein S is to be assayed.

Accordingly the final assay media for a series of samples which are to be compared contain sample and substrate for APC, and optionally also in the preferred variants one or two, preferably two, substances that do not derive from the sample and that are selected from APC, Protein S or an inhibitor to Protein S and Factor V having APC-cofactor 2 activity or an inhibitor to this activity, with the proviso that one of the remaining substances is the entity to be assayed (i.e. APC, Protein C, Protein S or APC-cofactor 2 activity ).

The present method may comprise a) incubating in one or more step in an aqueous assay medium, the sample and a substrate for APC, said substrate being inherently present in the sample or added to the assay medium, and optionally further blood coagulation components inherently present in the sample or added to the assay medium, b) measuring the conversion of the substrate caused by APC during the incubation according to a), and c) correlating the measured value in a known manner to the activity to be determined; in which method, optionally, one or two, preferably two, substances are added to the assay medium of a) said substance(s) being selected from APC, Protein S or a Protein S inhibitor, and Factor V having anticoagulant activity or an inhibitor to said activity, with the proviso that one of the remaining substances APC, Protein S or APC-cofactor 2 activity is present in the sample and is the component, the functional activity of which is to be determined, for Factor V, the said activity being anticoagulant activity as cofactor to APC.

Illustrative of other components that may be present are coagulation enzymes and other blood factors enabling the measurement of the degradation of Factors Vₐ and/or VIIIₐ. These other factors may be added separately or may be present already in the sample. In case the sample contains Protein C, and APC is to be assayed, an activator for Protein C must be added. In case the sample contains varying levels of coagulation factors (other than the one to be assayed) interfering with the assay reactions, one should secure excess of them (i.e. essentially constant levels in the assay media) in order to avoid inter-sample variations in the test results. For plasma samples constant levels may be accomplished by adding, in excess, normal plasma deficient in the entity to be assayed. The components to be added may also be in enriched or highly purified forms. It can be envisaged that addition of Factor VIII/VIIIₐ and/or forms of Factor V not expressing the APC-cofactor activity is suitable. Examples of forms that lack APC-cofactor activity are human Factor V deficient of the activity, Factor V from a species not normally expressing the activity (for instance bovine Factor V, and Factor V fragments expressing Factor V activity but not APC-cofactor 2 activity).

The addition of Protein S in the assay medium is done in order to avoid variations in the measured level caused by intersample variations in Protein S, when APC-cofactor 2 activity or Protein C is to be measured. When Protein S is to be measured, APC-cofactor 2 activity may be added for the same purpose. The main idea behind this is to keep the functional activity level of factors other than the one to be determined essentially constant in the assay media on an interrun basis. As previously indicated this may be accomplished by including into the assay media such factors in excess, for instance by adding normal plasma in excess, and/or by including functional excess of inhibitors for such factors, e.g. antibodies binding to epitope responsible for the activity of such factors. Thus, a monoclonal antibody specific to the epitopes responsible for the APC-cofactor activity of Protein S has been successfully included (HPS 54, Dahlbäck et al., J. Biol. Chem. 265 (1990) 8127-8235) in assay media for assaying APC-cofactor 2 activity. Similarly, functional inhibitors for APC-cofactor 2 activity, like the above-mentioned monoclonal antibodies, may potentially be included in assay media when Protein S is to be assayed.

According to the present invention the functional assays are suitably performed in presence of added Factor VIII/VIIIₐ.

The principles for the order of mixing, components to be added and the different measuring principles are well-known in the field. See the above-mentioned citations. This also includes that APC activity may be followed by substrates such as fibrinogen (clotting assays) and chromogenic substrates for coagulation enzymes, the activity of which are influenced by APC activity. Suitable chromogenic, fluorogenic and luminogenic substrates are thus thrombin and Factor Xₐ substrates.

The sample is normally plasma from an individual/patient, or the sample may be Factor V having APC-cofactor 2 activity, Protein C (APC) or Protein S, all of these derived from a manufacturing process, or standards to be used in the assay.

Native Factor V (abbreviated FV) produced through recombinant technology (rFV) may be used instead of FV purified from plasma as an adduct in diagnostic methods for Protein C/APC or Protein S, as a standard or control in assays for FV anticoagulant activity or as a therapeutic agent for administration to patients partially or completely deficient in APC-cofactor 2 activity. Alternatively recombinant variants or fragments of FV with modified expressions of pro- or anticoagulant activity may be utilized for the same purposes and also in adducts in methods for FV anticoagulant activity. Such modifications may be generated through mutations of the thrombin or APC cleavage sites in FV. In the former case the procoagulant activity, and in the latter case the anticoagulant activity of FV, is partially or completely lost. Furthermore such species, or suitable immunogenic peptide fragments thereof, may be used for preparation of monoclonal antibodies for diagnostic or therapeutic use.

In assays for APC-cofactor 2 activity utilizing Factors Vₐ and/or VIIIₐ as the APC substrate and factors from the sample to measure APC substrate conversion, the sensitivity towards APC activity is considerably increased in plasmas from patients on treatment with vitamin K antagonists, resulting in an enhanced prolongation of clotting time in certain clotting assays, especially APTT tests. The increased sensitivity towards APC activity may be explained by the lowered levels of vitamin K dependent proteins such as Factors IX, X and II. Since APC-cofactor 2 activity is not vitamin K dependent, it may therefore become possible to measure this activity in plasmas from such patients by exogenous addition to the assay medium of certain vitamin K-dependent protein(s), such as at least one of Factors IX, IXₐ, X and II, optionally combined with Protein S. These proteins may be added in form of heavy metal salt eluate, such as a barium citrate eluate (Dahlbäck, Biochem. J. 209(1983)837-46) or aluminium hydroxide eluate (Bertina et al, Thrombos Hemostas 51 (1984) 1-5) or as purified components before measuring the APC substrate conversion. If the plasma contains heparin (standard or of low molecular weight) it is suitable to neutralize this effect by adding excess of heparin, or by adding polybrene or Protamine, or the like, as heparin inhibitors to reduce interference on the assay results.

As stated above the present methods for determining functional activities of PC/APC or Protein S or Factor V anticoagulant activity are similar to methods described earlier, e.g. in the cited references, the disclosure of which is included herein by reference. Thus, a detailed description of these methods should not be required. In principle, however, such methods are based on measurement of conversion of a substrate, the rate of which can be directly or indirectly determined and related to the substance to be assayed, e.g. based on coagulation or chromogenic assays, suitably in presence of further components necessary to detect the conversion rate, which are inherently present in, or added to, the sample.

Such components may be comprised of a reagent that serves to introduce an activated coagulation factor that can be used for determination of the substrate conversion rate. This reagent leads to the presence of at least Factor IXₐ, and may be comprised of a certain coagulation factor or a reagent that activates the system via the intrinsic or extrinsic pathway. Accordingly, this reagent may be Factor IXₐ or Factor XIₐ (intrinsic pathway), Factor XIIₐ (intrinsic pathway), kallikrein (intrinsic pathway), a contact activator (intrinsic pathway) such as kaolin, celite or ellagic acid (intrinsic pathway), an APTT reagent (Activated Partial Thromboplastine Time; i.e. a reagent containing a phospholipid and a contact activator (intrinsic pathway)), tissue thromboplastin (PT-reagent, PT=Prothrombin time (extrinsic pathway)), tissue factor, Factor VIIₐ and Factor Xₐ.

Other components, that can be added, depend on the mode employed and may necessitate the inclusion of plasma protease inhibitors for enzymes other than the monitored one or of a fibrin polymerization inhibitor. Ca²⁻ may be in the form of a plasma soluble salt that provides the Ca²⁺ ion in free uncomplexed form, i.e. strong Ca²⁺ ion in free uncomplexed form. Such additional components suitably also include Factor VIII/VIIIₐ and Factor V/Vₐ.

The substrate for which the conversion rate is determined may be comprised of a synthetic substrate for an enzyme, the activity of which is influenced by activated Protein C, e.g. thrombin (=Factor IIₐ) and Factor Xₐ. Suitable synthetic substrates are water soluble and have preferably an oligopeptide structure with three, four or five amino acid residues and an amino terminal that is protected from being attacked by amino peptidases. The protection is accomplished either by a protecting group or by having a D-amino acid in the amino terminal In order to give a detectable response the carboxi terminal of a synthetic substrate is amidated with a group that specifically can be released and detected upon action of the relevant blood coagulation protease. The group to be released is selected among chromogenic, fluorogenic or chemiluminogenic groups and other analytically detectable groups. See further H. C. Hemker, “Handbook of synthetic substrates for the coagulation and fibrinolytic system”, Martinus Nijhoff Publishers, 1983, and J. Fareed et al, “Synthetic peptide substrates in hemostatic testing” in CRC Critical Reviews in Clinical Laboratory Sciencies Vol 19, Issue 2, 71-134 (1983). In case of samples other than plasma samples exogenous fibrinogen may be added as substrate.

In order to accomplish a specific result with respect to the substance to be determined, in some cases one should try to keep the plasma sample content of the final assay medium as high as possible. Accordingly, a plasma sample content in tests having good specificity could be >10%, in particular >20% or >35% (v/v). In other cases, however, an essentially lower content, i.e. below 10% (v/v), can be used.

B. Immune assays for APC-cofactor 2 activity

The antibody preparation of the invention will enable immune assays of APC-cofactor 2 activity. Such assays mean that anti-APC-cofactor 2 antibody is allowed to form an immune complex with Factor V having APC-cofactor 2 activity in the sample in an amount that is a qualitative or quantitative measure of the APC-cofactor 2 activity level in the sample. The samples may be the same as for functional assays.

The present invention is also concerned with reagents for use in assays of B and C.

Purified preparations comprising Factor V expressing the APC-cofactor 2 activity, which has been purified from plasma or prepared by recombinant technology, Protein C preparations, optionally in an activated form or combined with a Protein C activator, and Protein S preparations, which contain defined amounts of their respective factor may be used as a reagent or standard or control in the above-mentioned assays. The Protein C preparation may be combined with at least one vitamin K dependent coagulation factor selected from Factors IX, X and II, optionally combined with Protein S. Products and preparations for therapeutic use may also be obtained by recombinant technology. Furthermore, the present monoclonal antibodies may be obtained by recombinant technology, and essentially PCR-technology, which is well known, may be used to obtain such antibodies having desired specifity.

There are indications that information may be obtained about various Factor V mutations based on interactions between Factor V anticoagulant activity and Protein S. Methods may be designed to obtain such information in presence or absence of a suitable antibody. Such methods in presence of antibody may be used as a quantitative method for an analyte, such as Factor V anticoagulation activity and Protein S.

C. Hybridization assays

Recent results obtained just prior to the filing of this patent specification have shown in a conventional DNA-linkage study of a large family with inherited APC resistance that there is a strong linkage between a neutral polymorphism in the Factor V gene and expression of APC-resistance. This strongly suggests that mutation in the Factor V gene is the cause for APC-resistance.This is a conclusive evidence that nucleic acid hybridisation assays as well as nucleic acid sequencing can be used in conventional ways in order to detect individuals at risk for thrombotic evidents due to a low level of APC-cofactor 2 activity. Thus, these type of assays may be used for checking, in an individual the abnormal presence or absence of one or more nucleic acid fragment(s) and/or sequence(s) unique for the presence or absence of expression of a Factor V molecule either carrying APC-cofactor 2 activity or being deficient in this activity. The protocols and conditions are the same as normally applied for other genes, except for now using reagents specific for Factor V gene and optionally mutation(s) associated with APC-resistance or specific for normal Factor V gene. Any cell sample from the individual may be appropriate.

Furthermore, the present invention is concerned with Factor V, suitably human Factor V, capable of becoming activated to exert Factor Vₐ procoagulant activity but not capable of exerting anticoagulant activity, preferentially not anticoagulant activity as a cofactor to APC, said factor being in a substantially pure form.

Another aspect of the invention is related to Factor V, suitably human Factor V, capable of exerting anticoagulant activity, preferentially as a cofactor to APC, but not capable of expressing procoagulant activity of Factor Vₐ.

Such Factors can be purified from plasma with similar methods as to normal Factor V, or prepared by recombinant technology. Possible applications are in standards and as supplementing reagents, and for therapeutic use.

The present invention is further disclosed in the following experimental section with reference to the drawings. On these drawings:

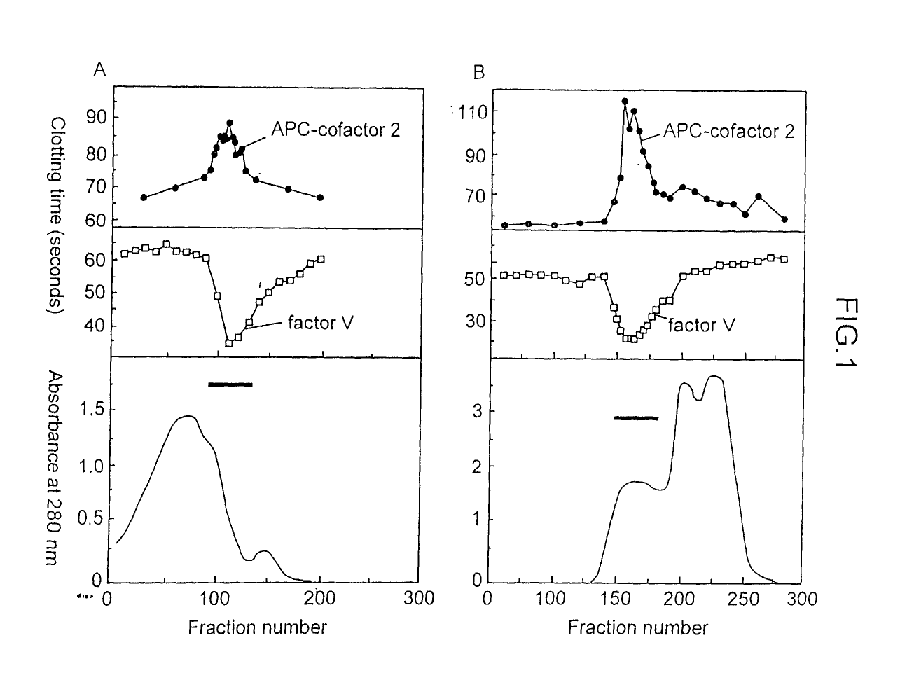

FIG. 1 illustrates chromatography on Q-Sepharose (A) and Sephacryl S-300 (B) of factor V and APC-cofactor 2 activity;

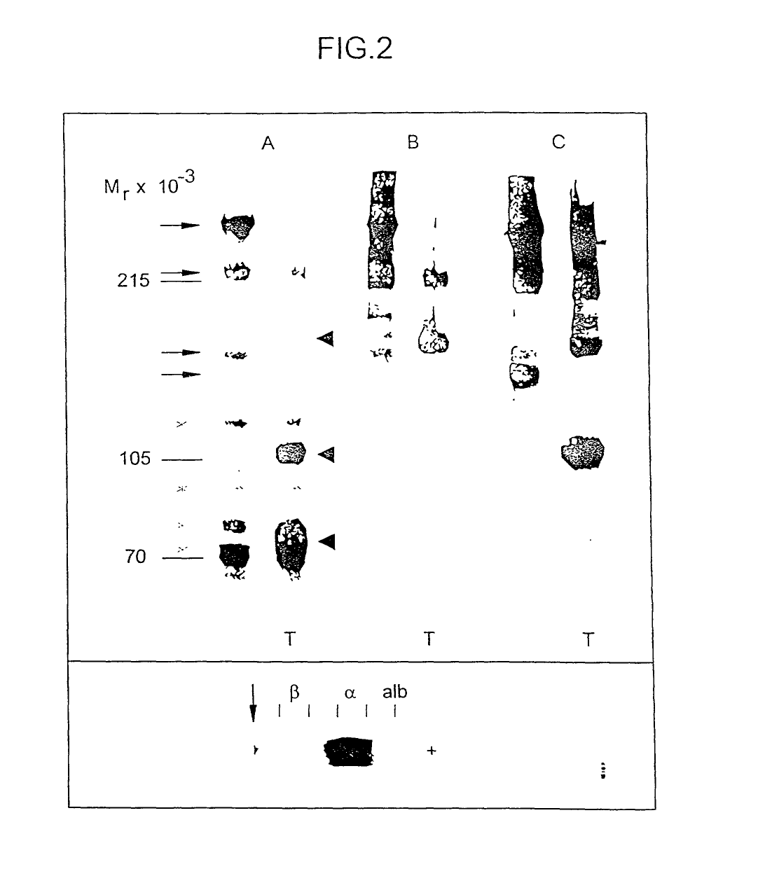

FIG. 2 illustrates the results from characterization of isolated APC-cofactor 2 activity/Factor V on SDS-PAGE, Western blotting, and agarose el electrophoresis;

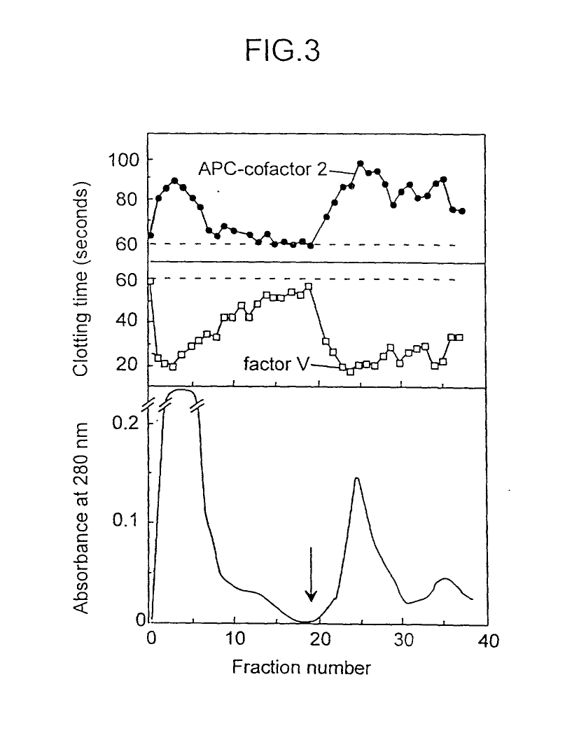

FIG. 3 illustrates copurification of APC-cofactor 2 activity and Factor V on monoclonal antibody affinity chromatography; and

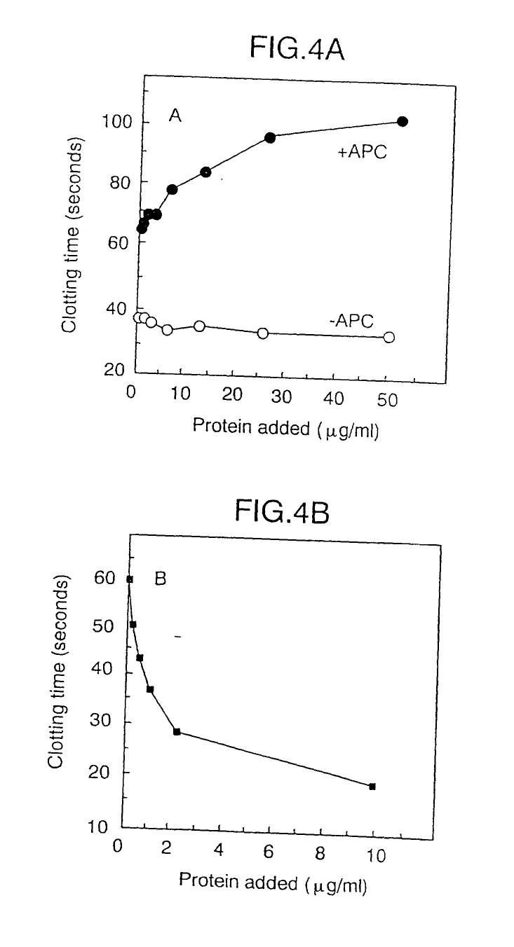

FIGS. 4A and B illustrate correction of APC-resistance by purified APC-cofactor 2 activity/Factor V.

### MATERIALS AND METHODS

Assay for APC-cofactor 2 activity

A modification of the recently described APC-APTT method (PCT/SE/9200781; and Dahlbäck et al., Proc. Natl. Acad. Sci. USA, 90 (1993) 1004-1008) was developed to measure APC-cofactor 2 activity during its purification. The method used plasma from an individual which had an inherited poor response to APC and fractions obtained from normal plasma which were tested for their ability to normalize the poor APC response. The assay which will be referred to as APC-cofactor 2 activity assay was performed as follows: 50 μl plasma demonstrating a poor response to APC (will be referred to as APC-resistant plasma) was incubated with 50 μl of the test fraction and 50 μl of an activated thromboplastin time (APTT) reagent (APTT-automated Organon Technica (USA)) for 5 minutes at 37° C. before coagulation was initiated by the addition of 5 μl of an APC-CaCl₂ mixture (if not indicated otherwise, 20 nM human APC in 10 mM Tris-HCl, 0,05 M NaCl, 30 mM CaCl₂, pH 7.5, containing 0.1% bovine serum albumin (BSA)), the coagulation time being measured. The presence of APC-cofactor 2 activity in a test sample is associated with an increase in clotting time.

Suitably, each sample is also analyzed in parallel without the addition of APC to the CaCl₂ solution and the APC-dependent prolongation of clotting time was calculated. To construct a dose-response curve for APC-cofactor 2 activity, the plasma deficient in APC-cofactor 2 activity was mixed with control plasma and used as test-plasma in the APC-APTT method. The anti-coagulant response of APC was related to the percentage of control plasma and the curve had an exponential shape. As it was unknown whether the plasma deficient in APC-cofactor 2 activity was completely devoid of Factor V expressing APC-cofactor 2 activity, the assay only provided a semi-quantitation of the cofactor in different fractions. However, the assay served the purpose of providing a means to follows the APC-cofactor 2 activity during its purification.

A factor V clotting assay was performed using factor V-deficient plasma as described previously (J. Clin. Invest. 66, 583-591 (1980)). The presence of Factor V activity resulted in a shortening of clotting time of the deficient plasma. In both APC-cofactor 2 activity assay and Factor V clotting assay the original clotting data have been shown rather than the results converted into units.

Electrophoretic, Immunological and Other Methods

Gradient (5-15%) polyacrylamide slab gel electroforesis in the presence of sodium dodecyl sulfate (SDS-PAGE) and Western blotting were performed using techniques previously described (J. Biol. Chem: 261, 9495-9501 (1986)). A specific rabbit polyclonal antiserum against Factor V was the kind gift of Dakopatts A/S. Data demonstrating the specificity of the antiserum have been reported previously (Blood 68, 244-249 (1986)). Rabbit polyclonal antibodies were raised against the isolated heavy and light chain fragments of bovine Factor V (J. Biol. Chem. 261. 9495-9501 (1986)). Monoclonal antibodies were raised using standard methods, as previously described in detail (J. Biol. Chem. 265, 8127-8135 (1990)). The purified protein in the S-300 pool was used as antigen in the immunization of Balb/c mice. Seventeen different antibodies were obtained and their reactivities tested with Western blotting. Approximately 20 mg of an antibody designated Master 30 was coupled to 4 ml Affigel 10 (Biorad) in accordance with the manufacturer's instructions. IgG-fractions of the polyclonal antisera against human Factor V and the bovine Factor V fragments were also coupled to Affigel (approximately 5 mg/ml).

### Example 1

Purification of APC-Cofactor 2 Activity

All manipulations of samples were performed on an ice bath; chromatographies and centrifugations were run in the cold room, suitably at 4° C. Blood-collection: Human freshly frozen (−70° C.) citrated plasma was obtained from the local blood bank. The frozen plasma (2.3 L) was thawed at 37° C. and the following protease inhibitors were added: phenyl methane sulfonyl fluoride (PMSF) (1 mM), drisopropylfluorophosphate (DFP) (1 mM), Soy bean trypsin inhibitor (50 mg/L), Trasylol (aprotinin) (1.5 mg/L which is equal to 10 units/ml), and benzamidine (10 mM). The plasma (kept on an ice-bath) was subjected to barium-citrate adsorption as previously described (Dahlbäck, Biochem. J. 209 (1983) 837-846) and the barium-adsorbed plasma was subjected to fractionated polyethylene glycol precipitation (PEG 6000) (8% w/v) by the addition of solid PEG. The APC-cofactor activity was recovered in the 8% PEG supernatant. The 8% PEG supernatant was diluted with an equal volume of 10 mM benzamidine and then mixed with Q-Sepharose (Pharmacia LKB Biotechnology, Uppsala, Sweden) equilibrated in 20 mM Tris-HCl, 0.1 M NaCl, 1 mM CaCl₂, pH 7.5, comprising 10 mM benzamidine. After 1 h of gentle mixing, the gel was collected in a Büchner funnel and washed with A, 3 L equilibration buffer, B, 1 L equilibration buffer with 0.1% Tween 20 and C, 2 L equilibration buffer containing 0.15 M NaCl instead of 0.1 M NaCl. The gel was then packed in a column (5 cm diameter) and the absorbed proteins were eluted with a linear gradient of NaCl (0.15-0.5 M NaCl in 20 mM Tris-HCl, 1 mM CaCl₂, 10 mM benzamidine, pH 7.5, 1.5 L in each gradient vessel). The flow rate was 330 ml/h and 11 ml fractions were collected. Fractions were analyzed for APC-cofator 2 activity and Factor V activity in 1/10 dilutions (FIG. 1).

Fractions were pooled as indicated by the horizontal bar and subjected to (NH₄)₂SO₄ precipitation (70% saturation). The precipitate was collected by centrifugation, dissolved in a minimal volume of 20 mM Tris-HCl, 0.15 M NaCl, 1 mM CaCl₂, pH 7.5, containing 10 mM benzamidine, 1 mM DFP, and 1 mM PMSF and applied to a column (2.5 cm×93 cm) with Sepharcryl S-300 (Pharmacia) equilibrated in the same buffer but without DFP and PMSF. The column was run at 10 ml/h and 1.2 ml fractions were collected. The fractions were analyzed with APC-cofactor 2 activity assay and Factor V assay, at 1/10 dilutions (FIG. 1). Fractions were pooled as indicated by the horizontal bar and stored at −70° C.

### Example 2

Affinity Chromatography Using Monoclonal Antibodies

The protein obtained in Example 1 from an S-300 chromatography (in the illustrated run approximately 6 mg in 20 mM Tris-HCl, 0.1 M NaCl, 2 mM CaCl₂, pH 7.5) was applied to a column (0.75 cm×7.5 cm) of Affigel with immobilized monoclonal antiboy designated Master 30 and obtained in example 3, the column and protein being equilibrated in 20 mM Tris-HCl, 0.1 M NaCl, 2 mM CaCl₂, pH 7.5. After washing the column until absorbance or the eluate reached zero, bound proteins were eluted with 50 mM diethanolamin, 2 mM CaCl₂, pH 10.6. The pH of the eluate was immediately neutralized with 3 M Tris-HCl, pH 7.5 (50 μl per 1 ml fraction). The fractions were analyzed (at 1/5 dilution) with APC-cofactor 2 activity assay and Factor V clotting assay. Active fractions were pooled, concentrated by ultrafiltration (YM10 membranes) and stored at −70° C. The purified APC-cofactor 2/Factor V was activated with thrombin as described previously (J. Clin. Invest. 66, 583-591 (1980)).

### Example 3

Preparation of Monoclonal Antibodies

The purified protein from Example 1, i e Factor V (APC-cofactor 2) was used as an immunogen for the immunization of Balb/c mice in accordance with a standard protocol. Splenic cells from said mouse were fused with cells of the Sp 2/0 Ag14 mouse myeloma cell line and selected in hypoxanthine-aminopterin-thymidin DMEM medium as disclosed by Köhler and Milstetn (loc. cit.)).

A solid phase enzyme-linked immunosorbent assay (ELISA) was used to detect antibodies produced against Factor V in antisera from the mice as well as to detect antibody-producing hybridic cells. In those assays, Factor V (10 μg/ml in standard coating buffer) was coated in wells on microtiter plates. Antisera from immunized mice and supernatants of the hybridic cell cultures were added in dilution to the wells and individual wells were assayed for the presence of antibodies bound to Factor V with the aid of an enzyme-labelled secondary antibody in a manner known per se.

Hybridic cells from positive wells, i e antibody-producing cells, were cloned by limiting dilution, subcloned and expanded. After implantation in the abdominal cavity of pristane pretreated mice, monoclonal antibodies were produced in ascitic fluid in large amounts.

Seventeen masters were obtained, all of which reacted with antigenic determinants on Factor V as shown in accordance with the Western blot method, the majority of these monoclonal antibodies (abbreviated Mab's) being directed to the same region of Factor V, viz the activation fragment comprised of the central 150 kDa region of Factor V.

One of these Mab's, designated Master 30, was used for the affinity purification of Factor V in accordance with Example 2.

### Example 4

In this example, the Mab's prepared in Example 3 were tested to determine their influence on coagulation activity and APC-cofactor 2 activity in plasma.

Increasing amounts of purified Mab up to 400 μg/ml were added to normal plasma and after incubation (15-30 minutes), the activity of Factor V was measured with a conventional Factor V assay based on coagulation analysis and the response to exogenous APC was determined according to the following.

Normal plasma samples comprising varying concentrations of Mab (10-400 μg/mL) were incubated with a commercial APTT reagent. (In the present tests Automated APTT from Organon was used. Similar results were obtained with the APTT reagent from COATEST APC Resistance, Chromogenix AB, Mölndal, Sweden.) After incubation for 5 minutes at 37° C. either 30 mM CaCl₂ (in 20 mM Tris-HCl, 50 mM NaCl, pH 7.5 comprising 0.1% bovine serum albumin (BSA)) or activated Protein C (APC) (about 2 μg/ml in 30 mM CaCl₂ dissolved in 20 mM Tris-HCl, 50 mM NaCl, pH 7.5 comprising 0.1% BSA) was added and the clotting times were recorded. The APTT assay was performed essentially as disclosed by Dahlbäck et al, PNAS 90 (1993) 1004-1008.

The presence of these Mab's did not affect the conventional APT time, i e the clotting time obtained for samples comprising added CaCl₂, at all, or only moderately, a clotting time of 40-45 seconds being observed. Two of the Mab's, designated Master 1 and Master 4, were, however, found to shorten the clotting time for samples, to which APC in a CaCl₂ solution had been added, (APC time).

The following clotting times were obtained:

| | |
|---|---|
| APC time in the absence of Mab’s | 110-120 seconds |
| APC time in the presence of Master 4 | 80-90 seconds. |

These results indicate an inhibition in part of the APC-cofactor 2 activity in plasma in the presence of Master 4. This partial inhibition activity of Master 4 was found to be dependent on the added amount, maximal inhibition being obtained when 50-100 μg of Master 4 per ml plasma were added. Master 4 has been deposited as stated above.

The results from the above tests are discussed belong with reference to FIGS. 1-4 (A,B). More specifically,

FIG. 1 illustrates chromatography on Q-Sepharose (A) and Sephacryl S-300 (B) of factor V and APC-cofactor 2 activity. On both columns, the elution profile of APC-cofactor 2 activity (upper sections) coincided with that of Factor V (middle sections). Factor V activity was demonstrated as a shortening of clotting time of Factor V-deficient plasma, whereas APC-cofactor activity was associated with an APC-dependent prolongation of clotting time of APC-resistant plasma. The fractions were pooled as shown by the horizontal bars.

FIG. 2 illustrates the results from characterization of isolated APC-cofactor 2/Factor V on SDS-PAGE, Western blotting, and agarose gel electrophoresis. The pool from the S-300 column obtained in Example 1 was analyzed by SDS-PAGE, before and after incubation with thrombin. The gels were either stained with Coomassie blue (A) or subjected to Western blotting using monoclonal antibody (Master 30) obtained in Example 3 (B) or polyclonal (C) antibodies. Samples applied to the SDS-PAGE were reduced; approximately 20 μg protein was applied to each lane in the protein-stained gel, whereas approximately 1 μg was applied to each of the lanes used for Western blotting. Lanes with thrombin-cleaved protein are marked T. Positions of molecular weight markers are given to the left, Factor V-related polypeptides are marked with arrows, whereas fragments formed by thromnbin (J. Biol. Chem. 257, 6556-6564) are indicated by arrowheads. The 150 kDa fragment staines poorly with Coomassie, but is readily observed on Western blotting. Intermittently observed bands are denoted by asterisks. The S-300 pool was also analyzed by agarose gel electrophoresis (bottom section). The positions of albumin (alb), α₁, α₂, β₁ and β₂ bands of a plasma control are indicated by vertical lines.

FIG. 3 illustrates copurification of APC-cofactor 2 activity, and Factor V on monoclonal antibody affinity chromatography. The S-300 pool was applied to monoclonal antibody (Master 30) affinity chromatography. As the binding capacity of the column was exceeded, most of the protein passed through the column. After washing the column, the bound protein was eluted with high pH (start of elution indicated by arrow). Fractions were analyzed with both APC-cofactor 2 activity and Factor V assay, Factor V activity was associated with a shortening of clotting time of Factor V-deficient plasma, whereas APC-cofactor activity gave an APC-dependent prolongation of clotting time of APC-resistant plasma. The two dashed lines represent clotting times of buffer controls.

FIGS. 4. A-B illustrates correction of APC-resistance by purified APC-cofactor 2/Factor V. Affinity purified APC-cofactor 2/Factor V (at indicated concentrations in a volume of 50 ul) was mixed with APC-resistant plasma (50 ml). The mixtures were then tested in the APC-cofactor 2 activity assay (A) with (●) and without (◯) APC in the CaCl₂-solution, and in the Factor V assay (B). Each point represents the mean of duplicate measurements.

### RESULTS

APC-cofactor 2 activity was analyzed with a biological assay using plasma from an individual (designated AS-plasma) with APC-resistance as test plasma, and a procedure was devised for purification of APC-cofactor 2 from normal plasma. The first step in the procedure was barium-citrate absorption, which removed the vitamin K-dependent proteins including proteins C and S. The barium-citrate eluate had no APC-cofactor 2 activity. On fractionation of the supernatant plasma with PEG 6000 precipitation, the APC-cofactor 2 activity was present in the 8% PEG supernatant, whereas the dissolved 0-8% PEG 6000 precipitate had no APC-cofactor 2 activity. The APC-cofactor 2 activity in the 8% PEG supernatant was purified first by anion exchange chromatography on a column with Q-Sepharose and then by gelfiltration on Sephacryl S-300 (FIG. 1). This purification protocol was very similar to a procedure of purification of coagulation Factor V (J. Clin. Invest. 66 583-591 (1980)), and Factor V was found in the same fractions of APC-cofactor 2 activity. The purification was performed at least 10 times with different modifications, and the elution profiles for Factor V and APC-cofactor 2 activity were consistently very similar. The protein in the S-300 pool expressed both Factor V and APC-cofactor 2 activities, and manifested characteristics previously reported for Factor V (J. Clin. Invest. 66 583-591 (1980)). Additional efforts to separate the two activities using several other chromatographic principles, such as Heparin Sepharose, Blue Sepharose and Wheat germ agglutinine Sepharose were unsuccessful (not shown), and APC-cofactor 2 activity was in fact found to purify together with Factor V on every chromatographic support, that was tried.

SDS-polyacrylamide gel electrophoresis of the protein in the S-300 pool yielded a 330 kDA band (corresponding to single chain Factor V) in addition to bands with molecular weights of approximately 220,000 and 130-150,000 (FIG. 2). These bands represented cleaved Factor V and, like the 330 kDa species, they reacted with a polyclonal antiserum against Factor V on Western blotting (FIG. 2). The 220 kDa band represented the C-terminal part of Factor V, including the 74 kDa light chain of Factor Vₐ and the larger (150 kDa) of the two activation fragments, and was recognized by an antiserum against the light chain of bovine Factor Vₐ (results not shown). The 130-150 kDa bands comprised the N-terminal part of Factor V (105 kDa heavy chain plus the smaller of the two activation fragments), and accordingly reacted with an antiserum against the bovine Factor Vₐ heavy chain (results not shown). Additional bands of lower molecular weights, which did not react with polyclonal Factor V antiserum on Western blotting were sometimes seen, but when present, their elution profiles (as judged by SDS-PAGE) on the S-300 chromatography did not correlate with the activity of Factor V or with APC-cofactor 2 activity. Incubation of the purified protein with thrombin yielded fragments characteristic for thrombin-cleaved Factor V and concomitantly the activity in the APC-cofactor 2 assay was lost suggesting APC-cofactor 2 activity only to be expressed by Factor V and not by Factor Vₐ. On Agarose gel electrophoresis, the purified protein migrated as a single species to an inter-alpha position (FIG. 2), and both Factor V and APC-cofactor 2 activities could be eluted from this position of the gel (not shown).

As Factor V is extremely sensitive to proteolysis, an abundance of protease inhibitors was included in the final protocol. When performed in the absence of protease inhibitors, the purification procedure resulted in a more degraded product lacking the 330 kDa species, but containing the 220 kDa and 130-150 kDa bands. This purified product expressed both Factor V and APC-cofactor 2 activities. Factor V requires calcium for its stability; and when calcium was not included in the purification, both Factor V and APC-cofactor 2 activities were gradually lost.

The protein in the S-300 pool was used as antigen in Example 3 in the production of monoclonal antibodies. Seventeen antibodies were obtained, and they were all found to react with the 330 kDa single chain Factor V as well as with the 220 kDa species, as judged by Western blotting (FIG. 2). After thrombin cleavage of Factor V, all antibodies reacted with the 150 kDa activation fragment (the larger of the two activation fragments).

One of the antibodies (Master 30) was immobilized on Affigel and used for affinity chromatography (FIG. 3). The S-300 pool was applied to the column. The protein that bound to the column was eluted and found to lane both Factor V and APC-cofactor 2 activities. The elution profiles of both activities coincided, but manifested considerable trailing. Other elution conditions such as using higher or lower pH, or denaturing agents were tried but were unsuccessful as they resulted in loss of both activities. The S-300 pool was also applied to columns with immobilized polyclonal antibodies against human Factor V or against bovine Factor Vₐ fragments. Both Factor V and APC-cofactor 2 activities were retained on the columns, but the denaturing conditions required to elute the round protein resulted in loss of both biological activities (results not shown).

Increasing concentrations of affinity purified APC-cofactor 2/Factor V were added to AS plasma and the anticoagulant response to APC tested. A dose-dependent increase in anticoagulant response to APC was observed (FIG. 4A), Approximately 25 mg/L, which is of the same order of magnitude as the normal plasma concentration of Factor V, was required to yield an APC-response of AS plasma comparable to that of normal plasma (clotting times in the presence of APC of 90-110 seconds). The affinity purified protein was also active in Factor V assay, as demonstrated by a shortening of the clotting time (FIG. 4B).

Assays For Components in the APC-Cofactor System

The following examples show that by keeping the levels constant of two of the components in the APC-cofactor system comprised of APC. Factor V having APC-cofactor 2 activity and Protein S, and varying the remaining one, different substrate conversion rates will be achieved. This implies that assays as outlined above for each of the components can be constructed. An assay employing plasma deficient in APC-cofactor 2 activity has been disclosed in the section Material and Methods.

### Example 5

Effect of APC-cofactor 2 in a Chromogenic Assay

The assay principle is based upon the monitoring of the degradation of FVIIIₐ by APC through the FIXₐ-dependent activation of FX in which system FVIIIₐ serves as an important cofactor to FIXₐ. Thus a decreased level of FVIIIₐ will result in a decreased generation of FXₐ, determined through the hydrolysis of a FXₐ-sensitive chromogenic peptide substrate.

I. 50 μL of a normal plasma dilution 1:20 in 50 mmol/L Tris-HCl buffer, pH 7.3, I=0.15 and 1% bovine serum albumin (BSA) containing highly purified FVIII concentrate (Octonativ M^{●}, Kabi Pharmacia AB, Stockholm Sweden), 2 IU/mL, was mixed with 50 μL bovine thrombin, 0.06 nka mL (activity vs. the substrate S-2238, (Chromogenix AB, Mölndal, Sweden)) for 30 s at 37° C.

II. Thereafter 100 μL of a reagent (R) mixture containing 40 mmol/L Tris-HCl, pH 7.3 and 0.15% BSA, CaCl₂, 12 mmol/L, and phospholipids, 30 μmol/L, as well as other components defined below, was added to he above mixture, followed by an incubation for 2 min at 37° C.

III. 25 μL was then subsampled from this mixture and diluted with 1000 μL 50 mmol/L Tris-HCl buffer, pH 7.3, I=0.15 with 0.2% BSA, followed by analysis of FVIII activity according to the COATEST^{●} FVIII assay principle (Chromogenix AB, Mölndal, Sweden).

IV. 200 μL of a reagent containing bovine FIXₐ and bovine FX (COATEST FVIII^{●}, Chromogenix AB, Mölndal, Sweden) and phospholipids, 30 μmol/L, was mixed with 100 μL of the diluted subsample and with 100 μL CaCl₂, 25 mmol/L. After 5 minutes incubation at 37° C., 200 μL of the chromogenic FXₐ-substrate S-2765 (Chromogenix AB, Mölndal, Sweden), 0.9 mmol/L was added. After further 3 min incubation at 37° C., the substrate hydrolysis was stopped by addition of 100 μL acetic acid, 20%, and the absorbance of the released chromphore pNA (p-nitroaniline) was read at 405 nm in a photometer.

In this assay system, the concentration of active FVIII in the sample is directly proportional to the absorbance. The content of supplemetary components in the different R-mixtures are:

A. None

B. APC, 0.4 μg/mL

C. APC, 0.4 μg/mL+APC-cofactor 2 activity, 0.3 U/mL

D. APC, 0.4 μg/mL+human Protein S, 1 μg/mL

E. APC, 0.4 μg/mL+human Protein S, 1 μg/mL+APC-cofactor 2 activity, 0.3 U/mL

Normal plasma contains approximately 10 ,μg/mL of free Protein S, hence the sample dilutions contributes with 0.05×0.05×10=0.025 μg in stage II, corresponding to one fourth of the added amount of purified human Protein S. The content of APC-cofactor 2 activity should be considered as an approximate estimation since no quantitative method yet exists.

Results:

| R- | Protein S. conc. | | Effect of APC-Cofactor 2 activity |
|---|---|---|---|
| mixture | in stage II, μg/mJ. | A 405 | on APC activity expressed as 405 |
| A | 0.125 | 0.678 | |
| B | 0.125 | 0.623 | |
| C | 0.125 | 0.509 | −0.114 (C-B) |
| D | 0.625 | 0.559 | |
| E | 0.625 | 0.389 | −0.170 (E-D) |

Thus the results show that addition of APC-cofactor 2 activity enhances the activity of APC at both levels of Protein S illustrated as a decrease in the FXₐ-generation, i.e. an increased rate of inactivation of FVIIIₐ in stage II.

### Example 2

Effect of APC-Cofactor 2 Activity in a Clotting Assay

Cofactors FVₐ and FVIIIₐ are involved in the generation of thrombin, the enzyme responsible for fibrin formation. These cofactors are degraded by APC and hence the activity of APC is illustrated in a clotting assay as a prolongation of the time needed for generation of the fibrin clot. Since Protein C (PC) circulates as a proenzyme, activation of PC in the sample is accomplished by addition of the snake venom enzyme Protac C^{●} (Pentapharm, Basel, Switzerland). The following experiment was performed:

I. 10 μL FVIII concentrate (Octonativ M^{●}, Kabi Pharmacia AB, Stockholm, Sweden), 10 IU/mL, was mixed with 100 μL PC-deficient plasma, 100 μL APTT reagent, 25 μL Protac C^{●, 1.5} U/mL, and 25 μL of a reagent (R) mixture containing 50 mmol/L Tris-HCl, pH 7.5, I=0.15, 0.2% BSA and further components defined below, was added to the above mixture. The complete mixture was incubated for 4 min at 37° C.

II. 100 μL CaCl₂, 22 mmol/L, was then added to the above mixture and the time needed for clot formation at 37° C. was recorded.

Supplementation in R-mixtures:

A. None

B. PC, 2 μg/mL

C. PC, 2 μg/mL,+APC-cofactor 2 activity, 2.6 U/mL

D. PC, 4 μg/mL

E. PC, 4 μg/mL,+APC-cofactor 2 activity, 2.6 U/mL

F. APC-cofactor 2, 2.6 U/mL

The Protein C deficient plasma contributes with the other plasma proteins involved in the clotting process and also with Protein S, a cofactor for APC. The final concentration of APC-cofactor 2 activity in stage I is approximately 0.2-0.3 U/mL (see above).

Results:

| | Concentration of | | Prolongation of APC |
|---|---|---|---|
| R- | PC in stage I, | | Activity due to |
| Mixture | μg/mL | Clotting time, s | APC-Cofactor 2 , s |
| A | 0 | 42.3 ± 0.7 (n = 5) | |
| B | 0.2 | 62.7 ± 1.2 (n = 5) | |
| C | 0.2 | 71.4 ± 1.6 (n = 3) | 8.7 |
| D | 0.4 | 79.3 ± 2.9 (n = 5) | |
| E | 0.4 | 1.045 ± 8.6 (n = 3) | 25.2 |
| F | 0 | 45.9 | |

Thus, the experiments clearly shows that addition of APC-cofactor 2 activity enhances the APC activity, expressed as an increased prolongation of the clotting time. The effect per se of the addition of the APC-cofactor 2 preparation in the absence of PC is only minor.

1. A method for determining in a sample the functional activity of a blood coagulation component, the said activity of which can be related to the conversion of a substrate specific for activated Protein C (APC); said method being an assay for components involved in the Protein C anticoagulant system, Protein C, activated Protein C, Protein S or anticoagulant Factor V being assayed, said method comprising measuring in an assay medium, containing the sample and a substrate for APC, the conversion of the substrate caused by APC and correlating the measured value in a known manner to the activity of the component to be to determined; in which method, optionally, one or two, preferably two, substances are added to the assay medium, said substance(s) being selected from APC, Protein S or an inhibitor that blocks sample derived Protein S activity, and Factor V, having anticoagulant activity or an inhibitor that blocks the same sample derived activity; with the proviso that one of the remaining substances, i.e. APC, Protein S and Factor V having anticoagulant activity is present in the sample and is the component, the functional activity of which is to be determined, for Factor V, the said activity being anticoagulant activity as cofactor to APC.

2. The method of claim 1, wherein the anticoagulant activity of Factor V as a cofactor to APC is determined, optionally in the presence of added Protein S, or an inhibitor that blocks sample derived Protein S activity.

3. The method of claim 1, wherein Protein C, after activation to APC, or APC is determined, at least one of Factor V, having APC-cofactor activity or an inhibitor that blocks the same sample derived activity, and Protein S or an inhibitor that blocks sample derived Protein S activity being added.

4. The method of claim 1, wherein Protein S is determined, at least one of Factor V having APC cofactor activity or an inhibitor that blocks the same sample derived activity, and APC being added.

5. The method of any of claims 1-4, wherein Factor VIII and/or VIIIₐ is added to the assay medium.

6. The method of claim 1, wherein the anticoagulant activity of Factor V as a cofactor to APC is determined, optionally in the presence of added Protein S or the said Protein S inhibitor, the substrate for APC is comprised of Factor Vₐ and/or Factor VIIIₐ and the sample is derived from an individual, coagulation proteins of the sample being utilized to measure APC substrate conversion; with the proviso that when the sample is derived from an individual on therapy with vitamin K antagonists or otherwise deficient in vitamin K dependent coagulation factors, the activities of such vitamin K dependent factors in the sample are modified by addition of at least one vitamin K dependent coagulation factor in activated or unactivated form, optionally in combination with Protein S.

7. The method according to any of claims 1-6, wherein the functional level of each selected substance added to the assay medium is essentially constant in the assay media of samples to be compared.

8. The method according to claim 7, wherein the essentially constant level is achieved by including into the assay medium a functional excess of the selected substance compared to the level provided by the sample.

9. The method of claim 7, wherein as the inhibitor, an antibody is used binding specifically to an epitope that is associated with the activity of APC, Protein C, or Protein S, or anticoagulant activity of Factor V as cofactor to APC.

10. The method according to any of claims 1-9, wherein the sample is a blood or blood derived sample, such as a plasma sample.

11. The method according to any of claims 1-10, wherein the one or two selected substance(s) has/have been provided in form of plasma deficient in the substance to be assayed.

12. The method according to any of claims 1-11, wherein the found level of the substance assayed is used to diagnose a blood coagulation disorder in the individual from which the sample is derived.

13. A method for diagnosing a blood coagulation/anticoaoulation disorder, preferably a thromboembolic disorder, or for determining predisposition therefor, in an individual, preferably a mammal, such as a human being, said method comprising determining the level of a blood component expressing anticoagulant activity in a sample, preferably a blood or blood derived sample, such as plasma, derived from said individual, said blood component being comprised of Factor V, an abnormal level indicating manifestation of, or predisposition for, said disorder, for a decreased level said disorder preferably being a thromboembolic disorder.

14. The method of claim 13, wherein the level of anticoagulant activity of Factor V as a cofactor to APC is determined.

15. The method of claim 14, wherein the anticoagulant activity is measured in accordance with the method of claim 2 adapted for determining anticoagulant activity of Factor V as cofactor to APC.

16. The method of claim 13, wherein Factor V having anticoagulant activity as a cofactor to APC is determined with an immunological method.

17. The method of claim 14, wherein the anticoagulant activity of Factor V as a cofactor to APC is determined in accordance with the method of claim 6.

18. The method of claim 15, wherein the anticoagulant activity of Factor V as a cofactor to APC is determined in the sample based on coagulation analysis, suitably with the method of claim 2, (i) one portion of the sample being incubated in absence of added APC but, optionally, in presence of further blood coagulation components required to enable measurement of substrate conversion by APC, and (ii) one portion of the sample being incubated in presence of added APC and, optionally, in presence of further blood coagulation components required to enable measurement of substrate conversion by APC; the clotting time being used, optionally after suitable conversion, and results below an established cut-off value based on clotting times, obtained in the same procedure for normal individuals, being indicative of a deficiency in anticoagulant activity of Factor V as cofactor to APC.

19. An antibody preparation reacting specifically with a region or site of Factor V that may carry an epitope associated with its anticoagulant activity as cofactor to APC.

20. The antibody preparation of claim 19, said preparation being monoclonal and comprising a definite number e.g. a number selected in the range of 1-5, of monoclonal antibodies having the specificity defined in claim 19.

21. Antibody preparation comprising polyclonal antibodies that recognize and selectively bind to Factor V, preferably to a region or site of Factor V associated with its anticoagulant activity as cofactor to APC, said region or site optionally comprising an epitope for said activity.

22. The antibody preparation of claim 20 comprising monoclonal antibodies, said antibodies being produced by mouse/mouse hybridoma cells, and preferably produced by the hybridoma cell line deposited in the European Collection of Animal Cell Culture under the provisional accession number AM-4-5-1 93120846.

23. A cell line producing [monoclonal] antibodies reacting specifically with a region or site of Factor V that may carry an epitope associated with its anticoagulant activity as cofactor to APC.

24. The cell line of claim 23, which is a hybridoma cell line.

25. The cell line of claim 24, which is the hybridoma cell line deposited in the European Collection of Animal Cell Culture under the provisional accession number AM-4-5-1 93120846.

26. Use of Factor V, subunits or fragments thereof, having anticoagulant activity as a cofactor to APC for the manufacture of a medicament or pharmaceutical preparation for enhancing, or restoring to normal, in an individual the anticoagulant activity of Protein C, APC, Factor V or Protein S, or any combination thereof.

27. Use according to claim 26, a medicament or pharmaceutical preparation for treatment of vascular diseases, preferably thromboembolic disorders, such as thrombosis and disseminated intravascular coagulation, being manufactured.

28. A plasma package preparation intended for the determination in vitro of anticoagulant activity of Factor V, as cofactor to APC, said preparation being comprised of human plasma, which has been made deficient in said anticoagulant activity, e.g. by immune depletion with respect to Factor V, capable of expressing said activity, said plasma optionally being supplemented with Factor V, e.g. bovine Factor V, from a species inherently lacking said anticoagulant activity, or with Factor Vₐ or said preparation being comprised of human plasma from one or more individuals, whose plasma is deficient in said activity.

29. The plasma package preparation of claim 28, wherein the deficient plasma is mixed with normal plasma, or with Factor V having anticoagulant activity, the mixture being capable to express the said anticoagulant activity at a suitable low level, e.g. for use as control plasma.

30. A plasma package preparation intended for determination in vitro of anticoagulant activity of Factor V as cofactor to APC, said preparation being a mixture of plasma from individuals having partial deficiency in Factor V anticoagulant activity, said mixture being capable to express said anticoagulant activity at a suitable low level, e.g. for use as control plasma.

31. A plasma deficient in anticoagulant activity of Factor V as cofactor to APC, said plasma being comprised of human plasma, which has been made deficient in said anticoagulant activity, e.g. by immune depletion with respect to Factor V capable of expressing said activity, said plasma optionally being supplemented with Factor V, e.g. bovine Factor V, from species inherently lacking said anticoagulant activity, or with Factor Vₐ, or being comprised of human plasma from one or more individuals, whose plasma is deficient in said activity.

32. The plasma of claim 31, wherein the deficient plasma is mixed with normal plasma, or with Factor V having said activity, or is a mixture of plasma from individuals having partial deficiency in Factor V anticoagulant activity, said mixture being capable to express said anticoagulant activity at a suitable low level, e.g. for use as control plasma.

33. Use of the antibody preparation of any of claims 19-22 to obtain an immune-depleted plasma package preparation of claim 28 or plasma of claim 31.

34. A Protein S preparation intended for the determination of anticoagulant activity of Factor V as cofactor to APC.

35. A Protein C preparation, optionally in activated form or combined with an activator for Protein C, intended for the determination of anticoagulant activity of Factor V as cofactor to APC.

36. A Protein C preparation according to claim 35, said preparation being combined with at least one vitamin K dependent coagulation factor selected from Factors VII, IX, X and II, optionally combined with Protein S.

37. Use of Protein C/activated Protein C or Protein S for the manufacture of a pharmaceutical composition intended for the treatment of a disorder related to functional disturbances in levels of anticoagulant activity of Factor V as cofactor to APC.

38. Factor V, suitably human Factor V, capable of becoming activated to exert Factor Vₐ procoagulant activity but not capable of exerting anticoagulant activity, preferentially not anticoagulant activity as a cofactor to APC, said factor being in a substantially pure form.

39. Factor V, suitably human Factor V, capable of exerting anticoagulant activity, preferentially as a cofactor to APC, but not capable of expressing procoagulant activity of Factor Vₐ.
